(19) United States
(12) Patent Application Publication
(10) Pub. No.: US 2005/0234320 A1
(43) Pub. Date: Oct. 20, 2005

(54) Method and apparatus for displaying medical information

(75) Inventor: Arun Balasubramanian, Bangalore (IN)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/085,997

(22) Filed: Mar. 22, 2005

(30) Foreign Application Priority Data
Mar. 31, 2004 (JP) 2004-103144

(51) Int. Cl. G06F 7/00; G06F 17/00; A61B 5/05

(52) U.S. Cl. 600/407; 600/410

(57) ABSTRACT

To provide an apparatus for displaying medical information, which facilitates recognizing any accompanying information, a medical image along with text information is displayed on a graphic display. Text information is provided as a voice presentation when the user points the text with a pointing device. The present invention allows hiding the text information on the screen, in which the text information is provided as the voice presentation when the user points the text field of the text information that should be there. The medical image may be taken by an X-ray CT or MRI apparatus.

### BACKGROUND OF THE INVENTION

The present invention is related to a method and apparatus for displaying medical information, more specifically to a method and apparatus for displaying a medical image in combination with text information on a graphic display device.

In a medical image network, the diagnosis is performed by a physician based on medical images downloaded from a server to a terminal (see Patent Document 1).

[Patent Document 1] Japanese Published Unexamined Patent Application No. H09-248280 (pp. 3 to 4, and FIG. 1).

A medical image is accompanied with information indicating the equipment used for the imaging and imaging conditions, and the accompanying information is displayed as texts on the terminal along with the medical image. The accompanying information may include any opinions and comments of physician, which are displayed in the same display screen as text information.

Although the text information is useful in diagnosis, the display thereof sometimes becomes impeditive for observing the image itself. In such a case, it can be appreciated that the display of text information is disabled intentionally for observing the image.

However, when the information display is disabled, the accompanying information cannot be seen as a natural conclusion. Thus, a frequent switch between the text display enabled and the display disabled. In addition, since the text information is displayed on the corner of screen, the visibility is not quite well.

### SUMMARY OF THE INVENTION

The object of the present invention is to provide a method and apparatus for displaying medical information, which facilitates the confirmation of the accompanying information.

(1) In an aspect of the present invention for solving the above-described problem, the present invention provides a method for displaying medical information, which displays a medical image in combination with text information in a graphic display device, including providing text information as the voice presentation when an operator points the text information with a pointing device.

(2) In another aspect of the present invention for solving the above-described problem, the present invention provides an apparatus for displaying medical information, which displays a medical image in combination with character information on a graphic display device, including display means for providing text information as the voice presentation when an operator points the text information with a pointing device.

It is preferable to allow disabling the display of text information while providing the text information as the voice presentation when an operator points the display area of the text information with a pointing device in a state in which the display of text information is disabled, in order to facilitate the confirmation of accompanying information when the text information is disabled.

It is also preferable that the medical image is taken by an X-ray CT apparatus for the purpose of facilitating diagnosis of the CT image. It is also preferable that the medical image is taken by an MRI CT apparatus for the purpose of facilitating diagnosis of the MRI image.

The present invention in aspects as have been described above, provides a method and apparatus for displaying medical information, which may facilitate the confirmation of accompanying information since the text information is provided as the voice presentation when an operator points the display area of the text information.

The present invention also provides a method and apparatus for displaying medical information, which enables non-display of only the text information, allowing facilitating the confirmation of accompanying information since the text information is provided as the voice presentation when an operator points the display area of the text information in a state in which the text information is not displayed.

Further objects and advantages of the present invention will be apparent from the following description of the preferred embodiments of the invention as illustrated in the accompanying drawings.

### BRIEF DESCRIPTION OF DRAWINGS

FIG. 1 is a schematic block diagram of an apparatus for displaying medical information in accordance with an exemplary embodiment of the best mode for carrying out the invention;

FIG. 2 is a flow diagram of the operation of the apparatus for displaying medical information;

FIG. 3 is a gray-scaled photograph indicative of an exemplary embodiment of displayed image; and

FIG. 4 is a gray-scaled photograph indicative of an exemplary embodiment of displayed image.

### DETAILED DESCRIPTION OF THE INVENTION

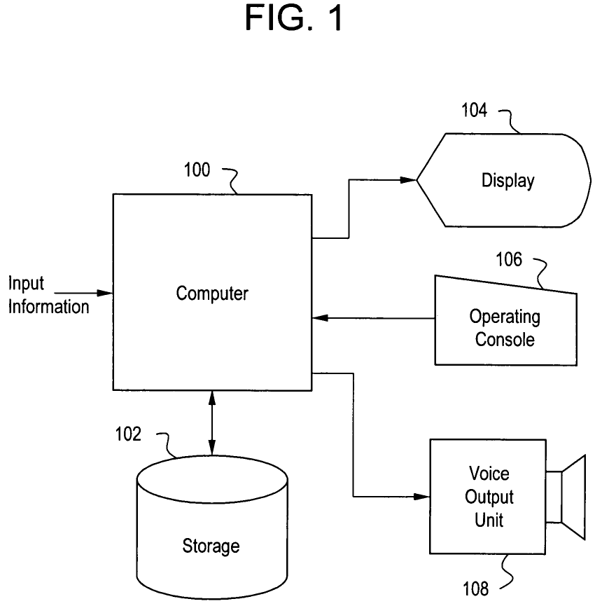

The best mode for carrying out the present invention will be described in greater details herein below in conjunction with the accompanying drawings. It should be understood that the present invention is not to be limited to the best mode for carrying out the invention. Referring to FIG. 1, there is shown a schematic block diagram of an apparatus for displaying medical information. The apparatus is an exemplary embodiment of the best mode for carrying out the present invention. The arrangement of this apparatus shows an exemplary embodiment of the best mode for carrying out the invention with respect to the apparatus for displaying medical information. The operation of this apparatus shows an exemplary embodiment of the best mode for carrying out the invention with respect to the method for displaying medical information.

As shown in FIG. 1, the apparatus includes a computer 100. The computer 100 is input with any information to be displayed. Input information is a combination of medical image and accompanying information, which may be downloaded for example from a server through a network. The input information composed of medical image along with accompanying information is conformable to for example a standard such as DICOM (digital imaging and communications in medicine).

The computer 100 has a storage 102. The storage 102 stores input image. The storage 102 also stores data and program for the computer 100. The data will be processed concerning the medical information display by executing the program stored in the storage 102 by the computer 100.

The computer 100 also has a display 104, an operating console 106, and an voice output unit 108. The display 104 may be a graphic display using for example a CRT (cathode ray tube) and the like, for displaying an image and any other information to be output from the computer 100. The voice output unit 108 performs voice output under the control of the computer 100.

The operating console 106 includes a keyboard equipped with a pointing device, such as a mouse, which is operated by the user (operator) in order to input instructions and information to the computer 100. The user is allowed to use the display 104, operating console 106 and voice output unit 108 for interactive operation of the apparatus.

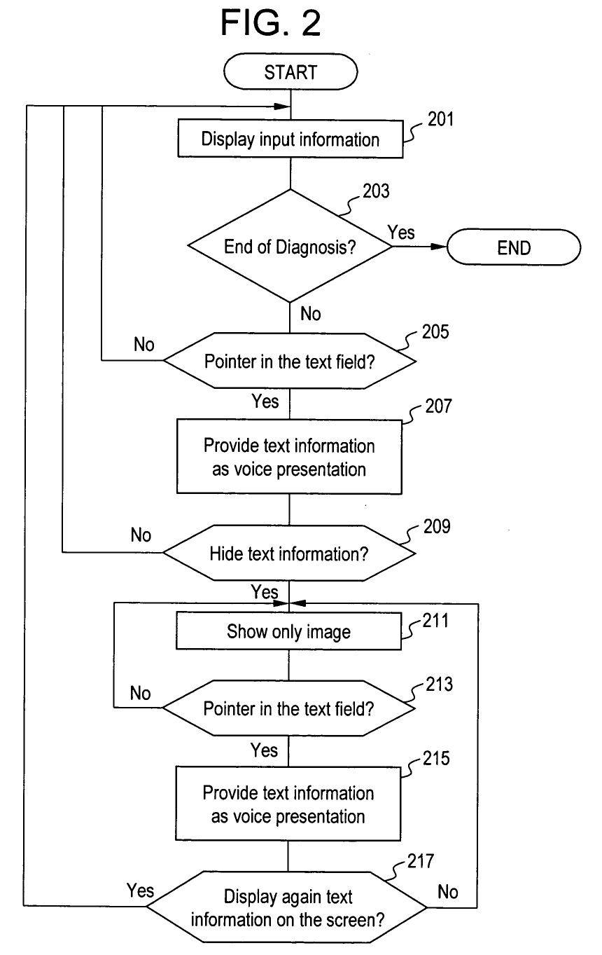

The operation of the apparatus will be described now. Referring to FIG. 2, there is shown a flow diagram of the operation of the apparatus. The operation of the apparatus is performed by the computer 100 executing a program stored in the storage 102.

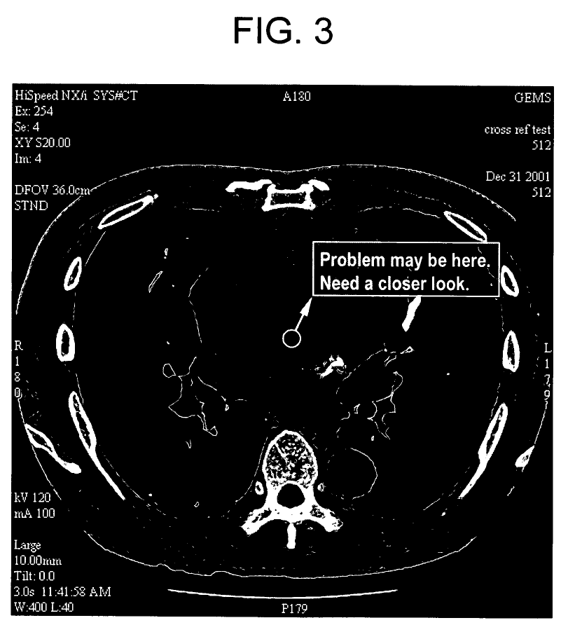

As shown in the figure, in step 201, input information is displayed. By this, input information such as that shown in FIG. 3 is displayed on the display 104. The input information may be a combination of a medical image taken by an X-ray CT apparatus, that is a tomographic image, and the accompanying information. The medical image is not limited to the image taken by the X-ray CT apparatus, but may also be that taken by an MRI (magnetic resonance imaging) apparatus, or any other suitable medical imaging apparatus including an X-ray imaging apparatus.

The tomographic image is gray-scaled. The accompanying information is binary image. The accompanying information may be character and numbers indicative of identification of imaging apparatus, imaging condition, and date. The accompanying information is also referred to as text information hereinafter. The text information includes numbers herein.

The text information is displayed for example in a corner of the display screen so as not to disturb the observation of tomographic image. The displaying position of text information is predefined. The display position of text image is referred to as text field, hereinafter.

The user of the apparatus, i.e., medical personnel such as physician, makes diagnosis based on the displayed information and inputs any opinions and comments on the screen as required. The text in the rectangular frame overlapped on the tomographic image indicates the text input.

In step 203, the process determines whether the diagnosis is completed or not. If completed, the operation of the apparatus terminates, while on the other hand if not completed yet, the process determines whether the pointer is within the text field or not in step 205. The pointer may be a UIF (user interface) that is operable by the pointing device, for example in the form of arrow.

If the pointer is not in the text field, then the process goes back to step 201 to continue to display input information. If the pointer is in the text field, then the process proceeds to step 207 to provide as the voice presentation the text information in the text field. The provision through voice is performed by the voice output unit 108. The voice output unit 108 is an exemplary embodiment of the display means in accordance with the present invention.

The provision as the voice presentation allows the user to recognize the contents of the text information by hearing. Each time the user points a desired text information, the text information is output as the voice presentation. This facilitates the user to recognize the contents of text information, which otherwise may be difficult to recognize due to small size appearance.

In step 209, the process determines whether to show or hide the text information. The text information can be arbitrarily hidden by the user. When hiding is instructed, in step 211, only the image is displayed. The instruction of hiding is input through the operating console 106. The operating console 106 is an exemplary embodiment of display disabling means.

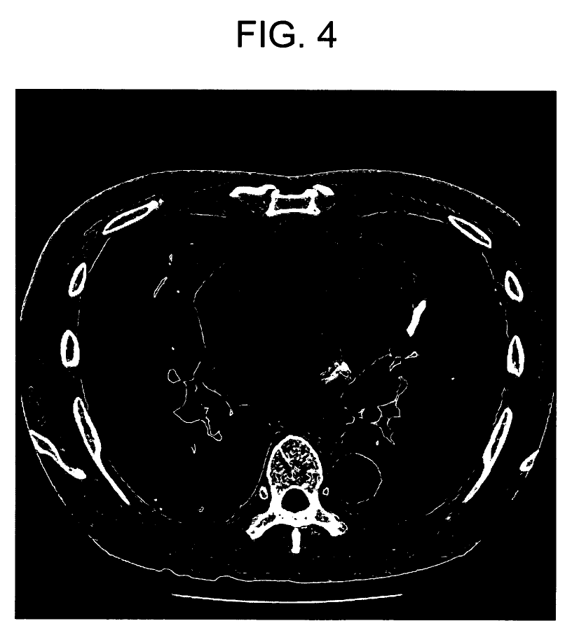

In such a manner, as shown in FIG. 4, a tomographic image without any text information accompanied is displayed on the screen. The image has no text information overlayed, thus will facilitate observation, with no obstructive text information as shown in FIG. 3. As a result, diagnosis will be facilitated based on the displayed image.

However, since no text information is displayed, the user cannot recognize any text information such as imaging conditions and the like. The following steps are provided so as to be able to recognize the contents of text information when the information is not displayed.

More specifically, in step 213, the process determines whether the pointer is in the text field. The text field can be pointed by the user even when no text information is displayed therein.

When the text field is pointed, in step 215, the text information that should be displayed in the text field will be provided as the voice presentation. Thus, the text information is not displayed but is output as the voice presentation. The user is allowed to recognize the contents of the text information owing to the voice presentation. The provision as the voice presentation is performed by the voice output unit 108. The voice output unit 108 is an exemplary embodiment of display means.

The user may proceed diagnosis while verifying through voice presentation the contents of the text information that should be displayed in the text field by pointing a desired text field. During this phase, the visibility of the image is held, because of no text information displayed on the screen.

In step 217, the process determines whether or not the text information display is to be shown. The text information display can be performed again on the screen by the user as needed, and if the user instructs to display again the information on the screen, the process goes back to step 201 based on the determination made in step 217. By so doing, the display of medical image in combination with accompanying text information as shown in FIG. 3 will be performed again on the screen. During the period in which the user does not instruct to redraw, the display of only medical image as shown in FIG. 4 is held.

Many widely different embodiments of the invention may be configured without departing from the spirit and the scope of the present invention. It should be understood that the present invention is not limited to the specific embodiments described in the specification, except as defined in the appended claims.

1. A method for displaying medical information, which displays a medical image in combination with text information in a graphic display device, comprising:
providing text information as a voice presentation when an operator points the text information with a pointing device.

2. A method of displaying medical information according to claim 1, wherein:
the display of only said text information is disabled; and
text information is provided as the voice presentation when an operator points the display area of text information in a state in which the text information is not displayed.

3. A method of displaying medical information according to claim 1, wherein:
said medical image is an image taken by an X-ray CT apparatus.

4. A method of displaying medical information according to claim 1, wherein:
said medical image is an image taken by an MRI apparatus.

5. An apparatus for displaying medical information, which displays a medical image in combination with character information on a graphic display device, comprising:
a display device for providing text information as the voice presentation when an operator points the text information with a pointing device.

6. An apparatus for displaying medical information according to claim 5, further comprising:
a display disabling device for disabling the display of only said text information; and
a display device for providing said text information as the voice presentation when an operator points the display area of said text information with a pointing device in a state in which the text information is not displayed.

7. An apparatus for displaying medical information according to claim 5, wherein:
said medical image is an image taken by an X-ray CT apparatus.

8. An apparatus for displaying medical information according to claim 5, wherein:
said medical image is an image taken by an MRI apparatus.
